(19) United States
(12) Patent Application Publication
(10) Pub. No.: US 2021/0008313 A1
(43) Pub. Date: Jan. 14, 2021

(54) Ventilator Setting Adjustment System

(71) Applicant: MediPines Corporation, Yorba Linda, CA (US)

(72) Inventors: Steve Lee, Anaheim Hills, CA (US); Richard Hinds, Yorba Linda, CA (US)

(21) Appl. No.: 16/810,077

(22) Filed: Mar. 5, 2020

Related U.S. Application Data
(60) Provisional application No. 62/814,184, filed on Mar. 5, 2019.

(51) Int. Cl. A61M 16/00; A61M 16/08

(52) U.S. Cl. CPC A61M 16/024; A61M 16/0858; A61M 16/085; A61M 16/0051; A61M 2205/583; A61M 2230/435; A61M 2230/42; A61M 2205/581; A61M 2230/432

(57) ABSTRACT

An automatic ventilator adjusting system has a three-way inline adapter coupled to 1) a breath sample line, 2) a ventilator (either invasive or non-invasive), and 3) a patient. The breath sample line is coupled to a Gas Exchange Monitor (GEM) and preferably has a female Luer lock end. Ventilator settings can be automatically set and/or adjusted using 1) an algorithm preferably having a feedback loop and 2) inputs including one or more of: gPaO₂™ (calculated arterial partial pressure of O₂ by GEM), oxygen deficit, gPaCO₂™ (calculated arterial partial pressure of CO₂), gPaCO₂™/gPaO₂™, PiO₂-PETO₂, TLC (Total Lung Capacity), FRC (Functional Residual Capacity), and Vd/Vt (deadspace ratio). Preferably, one or more of the inputs (e.g., gPaO₂™ gPaCO₂™, and oxygen deficit) are obtained non-invasively from a patient's normal breathing gas samples as calculated by MediPines Gas Exchange Monitor (GEM).

### CROSS-REFERENCE TO RELATED APPLICATIONS

This application claims priority to U.S. Provisional Application Ser. No. 62/814,184, filed on Mar. 5, 2019, which is incorporated herein by reference in its entirety.

### FIELD OF THE INVENTION

The field of the invention is ventilator setting adjustment.

### BACKGROUND

The following background discussion includes information that may be useful in understanding the present invention. It is not an admission that any of the information provided herein is prior art or relevant to the presently claimed invention, or that any publication specifically or implicitly referenced is prior art.

The mechanical ventilator (sometimes called respirator, breathing machine, ventilator, invasive ventilation etc.) is used primarily for treatment of patients in hospital setting, typically in Intensive Care Unit (ICU), Emergency Department or other area of the hospital where staff are specially trained to care for patients that have higher acuity than those in general patient areas. Mechanical ventilators are used for a wide variety of indications, but the vast majority of patients that require mechanical ventilation fall into two primary categories, those that have reduced drive to breathe (i.e., post-operative recovery, drug overdoses, neuromuscular disease or injury) and those for which their respiratory efforts are unable to adequately meet the demands of their body for oxygen or removal of carbon dioxide (i.e., Chronic Obstructive Pulmonary Disease, asthma exacerbation, severe congestive heart failure). Thus, ventilators provide a wide range of support for the work of a patient's breathing (moving air in and out of the lungs), from full support (in which the ventilator is doing all of the work of breathing for the patient) to no support (in which the ventilator is not doing any of the work of breathing for the patient.) Because patients can also have issues getting oxygen from within the lungs into their blood, extra oxygen can be added to the air given to the patient by the mechanical ventilator. The volumetric fraction of oxygen in the inhaled air given to the patient is referred to as FiO₂ (the fraction of inspired oxygen).

“Weaning” is a broad term that refers to the process of reducing the support of the mechanical ventilator for the patient. Generally speaking, patients are not taken off the breathing machine if the level of support they are receiving from the ventilator to off load the work of breathing is high, or if the FiO₂ the patients are receiving through the machine is high. The amount of time it takes to “wean” depends on the individual patient and there are very few standards as to how frequently a patient should be weaned. Once a patient has been weaned down enough for their setting to not be considered “high,” they generally undergo a process referred to as a Spontaneous Breathing Trial (SBT). During SBT, the support for the work of breathing is lowered to a level that essentially requires the patient to do all of the work of breathing. The trial should last from thirty minutes to two hours based on the patient's condition. Generally, they are done only once a day. At the end of the trial, the patient's efficiency of breathing and oxygenation status are evaluated, often with an arterial blood gas. In addition, clinicians review the work of breathing of the patient, as well as consider other issues that might arise if mechanical ventilation is discontinued. Unfortunately, many hospitals have low compliance conducting daily spontaneous breathing.

Some patients may be on a ventilator for only a few hours or days, while others may require the ventilator for longer. How long a patient needs to be on a ventilator depends on many factors. These can include the patient's overall strength, how well the patient's lungs were before going on the ventilator, and how many other organs are affected (e.g., the brain, heart, and kidneys). Some people never improve enough to be taken off the ventilator completely or at all.

In general, ventilator settings are usually manually input by a clinician using just their best judgment. The parameters that almost always set for patients on the breathing machine are: 1) Respiratory Rate (RR), the amount of breaths per minute the machine delivers or the patient takes on their own each minute; 2) Positive End Expiratory Pressure (referred to as PEEP) improves the volume of air in the lungs that is available for gas exchange; and 3) Partial Pressure of oxygen, which provides more oxygen to meet up with demand or diffusion limitation.

Some settings are specific to the mode of ventilation that the patient is in: 1) Tidal Volume (also known as VT; the amount of air pushed into the lungs on each normal breath, in a volume control mode); and 2) Inspiratory Pressure/Inspiratory Time (the amount of pressure and time that that the ventilator applies to the patient in Pressure control mode)

Positive end-expiratory pressure (PEEP) is used in a method of ventilation in which airway pressure is maintained above atmospheric pressure at the end of exhalation by means of a mechanical impedance, usually a valve, within the circuit. The purpose of PEEP is to increase the volume of gas remaining in the lungs at the end of expiration in order to decrease the shunting of blood through the lungs and improve gas exchange.

The level of positive end-expiratory pressure (PEEP) must be balanced such that excessive intrathoracic pressure (with a resultant decrease in venous return and risk of barotrauma) does not occur. However, currently there is no reliable way to set PEEP setting as it is based on clinician's judgment.

Thus, there is still a need for a reliable way to adjust ventilator settings, including PEEP, FiO₂, respiratory rate, tidal volume, inspiratory pressure, and inspiratory time.

Non-invasive ventilators provide ventilatory support to patients by providing two levels of pressure, commonly referred to as inspiratory positive airway pressure (IPAP) and expiratory positive airway pressure (EPAP) using a mask tightly fitted to the face or other interface. IPAP stands for Inspiratory Positive Airway Pressure and is the pressure delivered by the PAP device during inhalation. Expiratory positive airway pressure (EPAP) is positive airway pressure applied during the expiratory phase of mechanically assisted ventilation.

During non-invasive ventilation, the difference between IPAP and EPAP, often referred to as the pressure support, is the primary determinate of tidal volume, which changes with varying lung compliance and patient effort. The level of EPAP and delivered FiO₂ by the non-invasive ventilator is the primary determinant of oxygenation. Other common settings include respiratory rate and rise-time. Normally, these settings are currently manually set by the operator, and adjusted based on clinician's judgment.

The Vd/Vt ratio (i.e., Deadspace ratio, or deadspace to tidal volume ratio) is a useful measurement currently used by clinicians to determine the degree of lung damage, likelihood of successful liberation from the ventilator, and as an indicator associated with the risk of mortality. The current methods for calculating are based on the modified Bohr equation where Vd/Vt=(PaCO₂−PETCO₂)/PaCO₂. PaCO₂ is arterial blood CO₂ partial pressure measurement obtained from an invasive arterial blood gas method. PETCO₂ is an end tidal CO₂ which measures partial pressure of alveolar (lung level gas measurement) obtained non-invasively typically.

Information relating to monitoring of breathing gases can be found in co-pending U.S. application Ser. No. 15/814,902 to Lee et al., “System And Methods For Respiratory Measurements Using Breathing Gas Samples.” Information relating to breathing tubes for gas exchange monitors can be found in co-pending U.S. application Ser. No. 16/131,350 to Lee et al., “Breathing Tube for Gas Exchange Monitor.”

An important factor in setting the tidal volume on the ventilator is ensuring that a volume given to a patient is not so large that it distends or stretches the lungs in a way that will damage them. A very common method for manually setting the tidal volume for patients is to use their height, use a formula that converts the height into ideal body weight (IBW) and set the tidal volume based that measure (i.e., 6-8 ml/kg IBW). While widely used, there are serious limitations to this method. For instance, based on this method, two patients of the same height that are critically ill, will receive the same starting volume, without considering that their lungs may have very different available volumes due to conditions such as pulmonary edema, which is fluid inside the lungs, thus resulting in more stretching of the lungs of one of the patients.

A nitrogen washout/wash in procedure can be used to determine the useable total lung capacity (TLC) or Functional Residual Capacity (FRC). These measures, when done on patients that are on the mechanical ventilator are useful because tidal volume may be set proportional to the TLC or FRC, which are specific to the conditions of a patients lungs, rather than being set by other methods such as IBW, which do not take into account the individual variation of total usable lung size common in critically ill patients. The calculation is typically done by providing 10% decrease in inspired oxygen while measuring nitrogen concentration then oxygen is increased by 10% and the nitrogen concentration is measured. The dilutional effect on nitrogen is used to perform the volume calculation. Current methods for measuring these lung volumes with a nitrogen washout test are described in, “Multiple-breath nitrogen washout techniques: including measurements with patients on ventilators. C J Newth, P Enright, R L Johnson European Respiratory Journal September 1997, 10 (9) 2174-2185.

When patients can no longer maintain ventilation sufficient for the demand of their body, the result is respiratory failure. The current methodology to detect this type of failure is to look at the PaO₂ and PaCO₂ from an arterial blood gas, where PaO₂ is the partial pressure of arterial oxygen (O₂) and PaCO₂ is the partial pressure of arterial carbon dioxide (CO₂). If the PaO₂ is equal to or lower than the PaCO₂, respiratory failure and the need for further intervention are likely.

All extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

### SUMMARY OF THE INVENTION

The inventive subject matter provides apparatus, systems, and methods in which an automatic ventilator adjusting system has a three-way inline adapter, and ventilator settings can be set and/or adjusted using an algorithm and various inputs.

Contemplated inputs include one or more of the following: gPaO₂™ (calculated arterial partial pressure of O₂ by GEM), oxygen deficit, gPaCO₂™ (calculated arterial partial pressure of CO₂ by GEM), gPaCO₂™/gPaO₂™, PiO₂-PETO₂ (Oxygen Uptake), TLC (Total Lung Capacity), FRC (Functional Residual Capacity), and deadspace ratio Vd/Vt, wherein PiO₂ is the partial pressure of inspired oxygen and PETO₂ is the end-tidal partial pressure of oxygen (O₂). Preferably, one or more these inputs are non-invasively measured by MediPines Gas Exchange Monitor (GEM).

With the GEM mouthpiece adapted for use in-line to sample from the breathing circuit of a mechanical ventilator, placed beyond the wye of the ventilator circuit (between the end of the endotracheal tube and the bifurcation of the breathing circuit into an inspiratory and expiratory limb), outputs from the GEM can enter a closed feedback loop in the processor of the mechanical ventilator. Contemplated GEM outputs include: gPaO₂™ (calculated arterial partial pressure of O₂ by GEM), oxygen Deficit, gPaCO₂™ (calculated arterial partial pressure of CO₂ by GEM), gPaCO₂™/gPaO₂™, PiO₂-PETO₂, TLC and FRC output. The ventilator can then use those feedbacks to autonomously make changes to the ventilator settings. The operator may choose which ventilator settings are autonomously controlled, and which settings, if any, the user would like to control. gPaO₂™ is the calculated respiratory gas-based arterial partial pressure of oxygen by GEM. gPaCO₂™ is the calculated respiratory gas-based arterial partial pressure of carbon dioxide by GEM. Oxygen deficit is a difference between the end tidal partial pressure of oxygen, typically known as PETO₂, achieved during steady-state breathing minus gPaO₂™ and is a single measure of the degree of impaired gas exchange of the patient. gPaO₂™, gPaCO₂™ and oxygen deficit are preferably obtained non-invasively from a patient's normal breathing gas samples as calculated by the GEM device.

In preferred embodiments, the gPaO₂™ output signal can be an input signal to the ventilator to change the FiO₂, the positive end expiratory pressure (PEEP), the respiratory rate, the IPAP, EPAP, tidal volume, inspiratory time, inspiratory pressure, or any combination thereof, in order to achieve a target gPaO₂™. For example, if measured gPaO₂™ is lower than the target gPaO₂™ set by the operator, FiO₂ and/or PEEP is increased autonomously by the ventilator controlled by a processor running a computer algorithm. If measured gPaO₂™ is higher than target, then the algorithm would lower FiO₂ and/or PEEP. If that target cannot be obtained with in parameter limits that the operator has set, the ventilator will display a warning message to the operator and/or sound an audible alarm. Another example would be if the measured gPaCO₂™ is higher than the target gPaCO₂™ set by the operator, the pressure support, tidal volume and/or respiratory rate will be increased autonomously by the ventilator. If the measured gPaCO₂™ is lower than the target gPaCO₂™ set by the operator, the tidal volume and/or respiratory rate will be decreased autonomously by the ventilator. The expected level degree of increase and decrease is well understood and used in current practice by clinicians that manually adjust the ventilator settings currently. PEEP will be autonomously adjusted by the ventilator up or down to achieve the lowest oxygen deficit.

In some embodiments, the oxygen deficit output signal will be an input signal into a closed loop algorithm of the ventilator. The oxygen deficit can signal the ventilator to change the IPAP, EPAP, FiO₂, the positive end expiratory pressure (PEEP), or any combination thereof. In the case of PEEP or EPAP, the oxygen deficit can be provided into a closed loop algorithm to determine the optimum level of PEEP to apply to the patient and provide a signal of harmful over-inflation of the lungs.

In some embodiments, gPaCO₂™ output signal can be used to feed into a closed loop algorithm used by the ventilator to adjust pressure support, tidal volume, respiratory rate, FiO₂, PEEP, inspiratory flow, inspiratory pressure, inspiratory time, or any combination thereof to achieve a target gPaCO₂™. If that target cannot be obtained with in parameter limits that the operator has set, the ventilator will display a warning message and/or sound an alarm to the operator.

In some embodiments, the gPaCO₂™/gPaO₂™ ratio will be used to increase ventilatory support. For example, if the gPaCO₂™/gPaO₂™ ratio is greater than 1, the ventilator will increase support to the patient by increasing pressure support, respiratory rate and tidal volume, and peak flow rate, or combination thereof, to increase support while avoiding incomplete exhalation.

In some embodiments, the gPaCO₂™ and PETCO₂, obtained during steady state breathing, can be used to calculate a deadspace ratio Vd/Vt, using the equation Vd/Vt=(gPaCO₂™−PETCO)/gPaCO₂™. The final Vd/Vt value from the above equation can be used to adjust one or more settings of the ventilator. This input value can be used to adjust autonomously one or more ventilator parameters (e.g., PEEP) to a target Vd/Vt value established by the operator. For example, targeting a threshold Vd/Vt of ˜0.33 and PEEP can be raised up or down to achieve the threshold value. Preferably, the threshold Vd/Vt is set as low as possible (possibly below 0.33) depending on the physiologic response of the patient.

In some embodiments, the PiO₂-PETO₂ output signal can be an input signal into a closed loop algorithm of the ventilator used to adjust PEEP. For example, if the PiO₂-PETO₂ output exceeds a limit set by the operator, then the ventilator will autonomously and incrementally increase the PEEP to reduce difference.

In some embodiments, the TLC and/or FRC output signal can be created by decreasing the FiO₂ by 10%, measuring the combined PETO₂ and PETCO₂ outputs, thus generating an estimate of Nitrogen concentration (the remainder). The device will increase the FiO₂ back up 10% and the resulting estimate of nitrogen will be used to comparatively calculate the FRC of the patient.

In some embodiments, the TLC and FRC output signal can be used to feed into a closed loop algorithm that will set the tidal volume of the ventilator. For example, the tidal volume can be set based on a percentage of the TLC and FRC. A target range or limit can be set by the ventilator operator.

In some embodiments, the above parameters may also be used by the ventilator in a closed loop algorithm to determine if the ventilator may begin to reduce the work of breathing it does for the patient and may use the outputs to determine and switch the patient to a spontaneous breathing mode or spontaneous breathing trial (SBT). The ventilator can use the outputs to determine during a spontaneous breathing trial to determine when the ventilator should increase support to the patient. The ventilator can also use the outputs to determine that a patient has successfully passed a spontaneous breathing trial and alert the operator that the patient is ready for final evaluation for discontinuation of mechanical ventilation.

Some aspects of the inventive subject matter involves a method of adjusting a setting of a ventilator, including receiving a signal from a real-time monitoring system of a patient's exhalation, using the signal as an input to an algorithm to calculate an adjustment value for a setting of the ventilator; and automatically adjusting one or more settings of the ventilator based on the adjustment value. The signal can be one or more of the following: gPaO₂™, Oxygen Deficit, gPaCO₂™, PETCO₂, gPaCO₂™/gPaO₂™, PiO₂-PETO₂, TLC, and FRC output. The setting is can be one or more of the following: FiO₂, PEEP, respiratory rate, tidal volume, inspiratory time, and inspiratory pressure. The setting of the ventilator is adjusted in real-time or near real-time. Preferably, the real-time monitoring system is a MediPines Gas Exchange Monitor. In some embodiments, the algorithm can be a close loop algorithm. In some embodiments, the algorithm compares the signal with a target value of the signal. The contemplated method of adjusting a setting of a ventilator can further include producing a visual or audio message prompting a user to manually adjust the settings if the target value cannot be achieved within a reasonable period of time. The ventilator can be invasive, or non-invasive.

The step of automatically adjusting one or more settings of the ventilator can be one or more of the following: 1) increasing FiO₂ and/or PEEP if measured gPaO₂™ is lower than a target value, and lowering FiO₂ and/or PEEP if measured gPaO₂™ is higher than a target value; 2) increasing tidal volume and/or respiratory rate if measured gPaCO₂™ is higher than a target value, and decreasing tidal volume and/or respiratory rate if measured gPaCO₂™ is lower than the a target value; 3) increasing support to a patient if gPaCO₂™/gPaO₂™ ratio is greater than a limit set by the ventilator operator; 4) increasing the PEEP to reduce difference between PiO₂-PETO₂ output and a limit set by an operator, 5) decreasing the PEEP to reduce difference between PiO₂-PETO₂ output and a limit set by an operator; and 6) autonomously and incrementally increasing the PEEP. Increasing support can include one or more of the following: increasing respiratory rate and or tidal volume, increasing inspiratory flow rate, and avoiding incomplete exhalation.

In some embodiments, TLC output signal is created by temporarily decreasing FiO₂, and measuring the combined PETO₂ and PETCO₂ outputs. In some embodiments, FRC output signal is created by temporarily decreasing FiO₂, and measuring the combined PETO₂ and PETCO₂ outputs. In some embodiments, TLC and/or FRC output signal is fed into a closed loop algorithm that sets a tidal volume of the ventilator. In some embodiments, tidal volume is set based on a percentage of the TLC and FRC. In some embodiments, a target range or limit of tidal volume is set by the ventilator operator.

Some aspects of the inventive subject matter involve a system for adjusting ventilator settings including a processor configured to execute software instructions stored on a non-transitory storage medium. The software instructions to be executed include receiving a signal measured from a patient's exhalation; comparing the signal with a target value of the signal; calculating an adjustment value for a setting of the ventilator; and automatically adjusting the setting of the ventilator based on the adjustment value. The system can also include one or more of the following: 1) a ventilator coupled to the processor, 2) an inline Gas Exchange Monitor (GEM) adapter coupled to the ventilator, 3) a sample line coupled to the Gas Exchange Monitor adapter. Preferably, the sample line has a female Luer lock end. Also, preferably, the inline GEM adapter to ventilator circuit is coupled to the ventilator. The ventilator can be invasive, or non-invasive.

Some aspects of the inventive subject matter involve a method of adjusting a setting of a ventilator, including receiving a signal from a real-time monitoring system of a patient's exhalation; using the signal to calculate a deadspace ratio; using the deadspace ratio as an input to an algorithm to calculate an adjustment value for a setting of the ventilator; and automatically adjusting one or more settings of the ventilator based on the adjustment value. The signal can be one or more of following: gPaO₂™, Oxygen Deficit, gPaCO₂™, PETCO₂, gPaCO₂™/gPaO₂™, PiO₂-PETO₂, TLC, and FRC output. The deadspace ratio can be calculated using equation Vd/Vt=(gPaCO₂™−PETCO)/gPaCO₂™. The setting can be one or more of the following: FiO₂, PEEP, respiratory rate, tidal volume, inspiratory flow, inspiratory time, and inspiratory pressure. In preferred embodiments, the algorithm compares the calculated deadspace ratio with a target deadspace ratio set by an operator.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1A is a schematic diagram of an embodiment of a GEM adapter, having a breath sample line and an inline adapter to an invasive ventilator circuit.

FIG. 1B is an enlarged schematic diagram of the breath sample line and the inline adaptor to ventilator circuit in FIG. 1A.

FIG. 2 is a schematic diagram of an embodiment of a GEM adapter, having a breath sample line with adapter to a non-invasive ventilator circuit.

### DETAILED DESCRIPTION

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the context indicates the contrary.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

It should be noted that any language directed to a computer system should be read to include any suitable combination of computing devices, including servers, interfaces, systems, databases, agents, peers, engines, controllers, storage systems, or other types of computing devices operating individually or collectively. Computer systems may have full operating systems capable of executing complex processing tasks, or may be bare bones systems whose only function is to store, receive, and transmit data to memory storage units. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. In especially preferred embodiments, the various servers, systems, databases, or interfaces exchange data using standardized protocols or algorithms, possibly based on Fiber-Channel, PCIe Interface, NVMe, NVMe over Fabric, TCP, UDP, IP, HTTP, HTTPS, AES, public-private key exchanges, web service APIs, known financial transaction protocols, or other electronic information exchanging methods, including proprietary communication interfaces. Data exchanges preferably are conducted over a packet-switched network, the Internet, LAN, WAN, VPN, or other type of packet switched network. Computer software that is “programmed” with instructions is developed, compiled, and saved to a computer-readable non-transitory medium specifically to accomplish the tasks and functions set forth by the disclosure when executed by a computer processor.

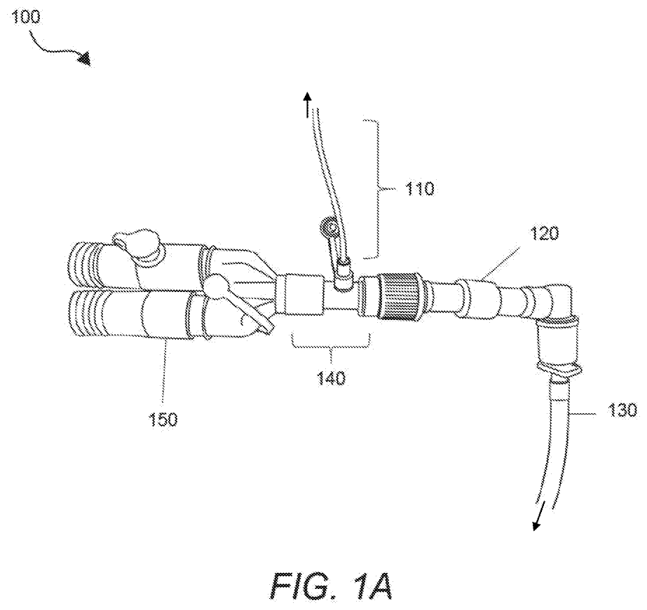

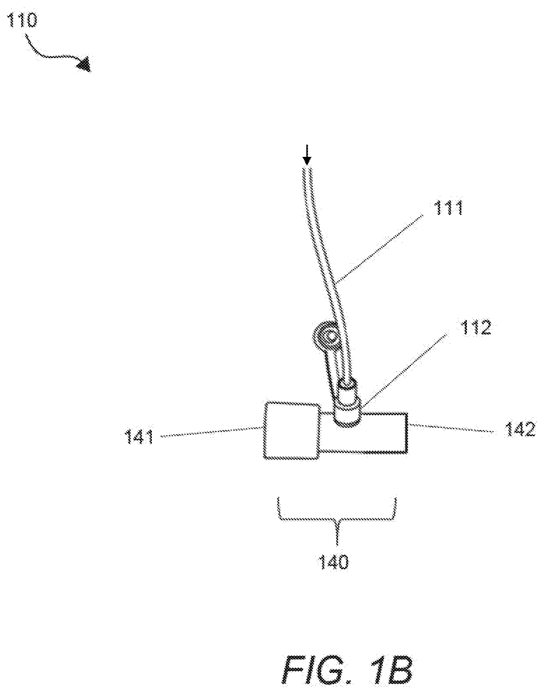

FIG. 1A is a schematic diagram of an embodiment of a GEM adapter, having a breath sample line 110 and an inline adapter 140 to an invasive ventilator circuit. The inline adapter 140 is coupled to a ventilator unit through wye 150. The breath sample line 110 is on one end coupled to the inline adapter 140, and on the other end coupled to a Gas Exchange Monitor (GEM). The inline adapter 140, through a series of standard respiratory tube adapters (e.g., 120), is coupled to an endotracheal tube 130 connected to a patient.

FIG. 1B is an enlarged schematic diagram of the breath sample line 110 and inline adaptor in FIG. 1A. The breath sample line 110 comprises a breath sample line 111 and a female luer lock end 112. The inline adapter 140 has a broader end 141 and a narrower end 142. In preferred embodiments, the broader end 141 has a 22 mm outer diameter (OD), and the narrower end 142 has a 15 mm outer diameter (OD).

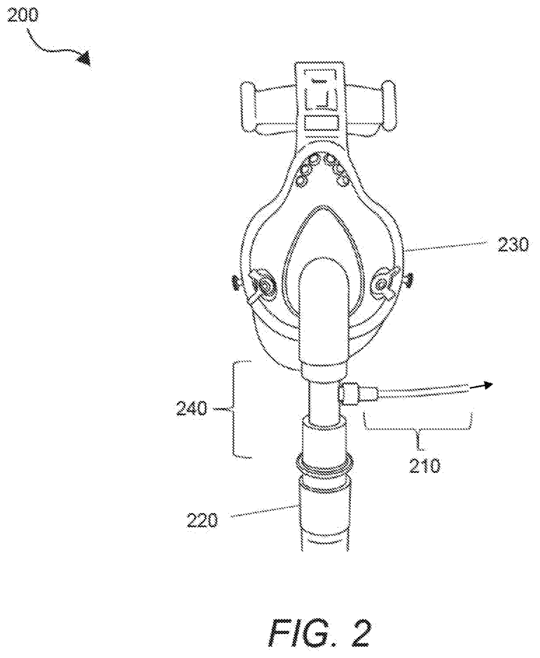

FIG. 2 is a schematic diagram of an embodiment of a GEM adapter, having a breath sample line 210 with an inline adapter 240 for a non-invasive ventilator circuit. The inline adapter 240 is coupled to a ventilator unit through a series of standard respiratory tube adapters (e.g., 220). The breath sample line 210 is on one end coupled to the inline adapter 240, and on the other end coupled to a Gas Exchange Monitor (GEM). The inline adapter 240 is coupled to a patient breathing mask 230 worn by a patient.

What is claimed is:

1. A method of adjusting a setting of a ventilator, comprising:
receiving a signal from a real-time monitoring system of a patient's exhalation;
using the signal as an input to an algorithm to calculate an adjustment value for a setting of the ventilator; and
automatically adjusting one or more settings of the ventilator based on the adjustment value.

2. The method of claim 1, wherein the signal is selected from the group consisting of: gPaO₂™ oxygen Deficit, gPaCO₂™, PETCO₂, gPaCO₂™/gPaO₂™, PiO₂-PETO₂, TLC, and FRC output.

3. The method of claim 1, wherein the setting is selected from the group consisting of: FiO₂, PEEP, respiratory rate, tidal volume, inspiratory time, and inspiratory pressure.

4. The method of claim 1, further comprising producing a visual or audio message prompting a user to manually adjust the settings if the target value cannot be achieved within a reasonable period of time.

5. The method of claim 1, wherein the algorithm comprises comparing the signal with a target value of the signal.

6. The method of claim 1, wherein the step of automatically adjusting one or more settings of the ventilator comprises at least one of 1) increasing FiO₂ and/or PEEP if measured gPaO₂™ is lower than a target value, and lowering FiO₂ and/or PEEP if measured gPaO₂™ is higher than a target value; 2) increasing tidal volume and/or respiratory rate if measured gPaCO₂™ is higher than a target value, and decreasing tidal volume and/or respiratory rate if measured gPaCO₂™ is lower than the a target value; and 3) increasing support to a patient when gPaCO₂™/gPaO₂™ ratio is greater than 1.

7. The method of claim 1, wherein the step of automatically adjusting one or more settings of the ventilator comprises at least one of: 1) increasing the PEEP to reduce difference between PiO₂-PETO₂ output and a limit set by an operator; 2) decreasing the PEEP to reduce difference between PiO₂-PETO₂ output and a limit set by an operator.

8. The method of claim 1, wherein at least one of TLC output signal and FRC output signal is created by temporarily decreasing FiO₂, and measuring the combined PETO₂ and PETCO₂ outputs.

9. The method of claim 1, wherein at least one of TLC and FRC output signal is fed into a closed loop algorithm that sets a tidal volume of the ventilator.

10. The method of claim 1, wherein at least one of volume, flow, and pressure signals from the ventilator is used to as an input to at least one of Vd/Vt calculation, FRC calculation, TLC calculation, and oxygen uptake calculation.

11. A system for adjusting ventilator settings, comprising:
a processor configured to execute software instructions stored on a non-transitory storage medium, wherein the software instructions comprise:
receiving a signal measured from a patient's exhalation;
comparing the signal with a target value of the signal;
calculating an adjustment value for a setting of the ventilator; and
automatically adjusting the setting of the ventilator based on the adjustment value.

12. The system in claim 11, further comprising a ventilator coupled to the processor.

13. The system in claim 11, further comprising a Gas Exchange Monitor adapter coupled to the ventilator.

14. The system in claim 11, further comprising a sample line coupled to the Gas Exchange Monitor adapter.

15. The system in claim 11, further comprising an inline GEM adapter to ventilator circuit coupled to the ventilator.

16. A method of adjusting a setting of a ventilator, comprising:
receiving a signal from a real-time monitoring system of a patient's exhalation;
using the signal to calculate a deadspace ratio;
using the deadspace ratio as an input to an algorithm to calculate an adjustment value for a setting of the ventilator; and
automatically adjusting one or more settings of the ventilator based on the adjustment value.

17. The method of claim 16, wherein the signal is selected from the group consisting of: gPaO₂™, oxygen deficit, gPaCO₂™, gPaCO₂™/gPaO₂™, PiO₂-PETO₂, TLC, and FRC output.

18. The method of claim 16, wherein the deadspace ratio (Vd/Vt) is calculated using an equation, wherein the equation comprises Vd/Vt=(gPaCO₂™−PETCO₂)/gPaCO₂™.

19. The method of claim 16, wherein the setting is PEEP.

20. The method of claim 16, wherein the algorithm comprises comparing the calculated deadspace ratio with a target deadspace ratio set by an operator.
